(19) United States
(12) Patent Application Publication
(10) Pub. No.: US 2014/0303264 A1
(43) Pub. Date: Oct. 9, 2014

(54) Process for Making and Using Cellulose-Containing Seaweed Residue and Products Made Therefrom

(71) Applicants: Zheng Tan, Princeton, NJ (US); Michael Sestrick, Yardley, PA (US); William Matakas, Downingtown, PA (US); Aaron Chip Venables, Yardley, PA (US)

(72) Inventors: Zheng Tan, Princeton, NJ (US); Michael Sestrick, Yardley, PA (US); William Matakas, Downingtown, PA (US); Aaron Chip Venables, Yardley, PA (US)

(73) Assignee: FMC CORPORATION, Philadelphia, PA (US)

(21) Appl. No.: 14/241,497

(22) PCT Filed: Aug. 31, 2012
(86) PCT No.: PCT/US12/53446

§ 371 Date: May 19, 2014

Related U.S. Application Data
(60) Provisional application No. 61/573,284, filed on Sep. 2, 2011.

(51) Int. Cl. C08B 15/00; A23G 9/34; A61K 47/38

(52) U.S. Cl. CPC C08B 15/00; A61K 47/38; A23G 9/34
USPC 514/781; 536/56; 127/34; 426/567; 426/539; 428/402

(57) ABSTRACT

The present invention is directed to a process comprising: (i) extracting ≧50% by weight of all carrageenan from a carrageenan-containing seaweed material to obtain a cellulose-containing seaweed residue; and (ii) purifying the cellulose-containing seaweed residue by at least one of hydrolysis or bleaching. The present invention is also directed to the cellulose obtained from the process, as well as products containing the cellulose-containing seaweed material.

### FIELD OF THE INVENTION

The present invention is directed to a process comprising (i) extracting ≧50% by weight of all carrageenan from a carrageenan-containing seaweed material to obtain a cellulose-containing seaweed residue; and (ii) purifying the cellulose-containing seaweed residue by at least one of hydrolysis or bleaching. The present invention is also directed to the cellulose-containing seaweed residue obtained in the process and products made therefrom.

### BACKGROUND OF THE INVENTION

Carrageenan is a commercially significant galactan polysaccharide found in certain red seaweed and constitutes the principal structure of the seaweed. All carrageenans contain repeating galactose units joined by alternating α1→3 and β1→4 glycosidic linkages and are sulfated to widely varying degrees. It is located within the cell wall and intercellular matrix of the plant tissue. The carrageenan content of commercially harvested seaweeds is generally between 30% and 80% by weight based on the seaweed dry weight.

The carrageenan manufacturing process typically involves significant hot water and/or alkali treatments of the seaweed so as to extract the carrageenan from the seaweed. Importantly, this carrageenan extraction process generates significant amounts of seaweed residue and the carrageenan extraction process affects the quality and type of the cellulose and polysaccharides in the seaweed. After carrageenan extraction, the quality and quantity of cellulose found in the seaweed residue has been considered to be low in value and, as a result, the seaweed residue has simply been considered waste material and disposed of as such.

The present inventors have unexpectedly found that the cellulose remaining in the seaweed residue (after carrageenan extraction) is contained in the seaweed residue in sufficient quantities to be commercially significant and that it possesses unexpected morphology and functionality. As a result, the purified cellulose-containing seaweed residue of the present invention may be turned into a value added side stream and be used in, for example, food, pharmaceutical, and health or consumer products, as well as industrial applications.

### SUMMARY OF THE INVENTION

The present invention is directed to a process comprising (i) extracting ≧50% by weight of all carrageenan from a carrageenan-containing seaweed material to obtain a cellulose-containing seaweed residue; and (ii) purifying the cellulose-containing seaweed residue by at least one of hydrolysis or bleaching. The present invention is also directed to the cellulose-containing seaweed residue obtained from the process, as well as products containing the cellulose-containing seaweed material.

### BRIEF DESCRIPTION OF THE FIGURES

FIG. 1 is a diagram of the invention showing steps that may, but are not required, to be performed in the carrageenan extraction step involving the “conventional extract process” (defined below).

FIG. 2 is a diagram of the invention showing different steps that may, but are not required, to be performed in the carrageenan extraction step involving what is typically referred to as the “semi-refined carrageenan process” (discussed below).

FIG. 3 is the cellulose-containing residue of the invention dispersed in deionized water at 2.6% solids. See Example 2.

FIG. 4 shows a rheology test for 1.9% (triangles) and 2.3% (squares) solid suspensions containing the cellulose-containing residue of the present invention. The results demonstrate that both suspensions showed a very high gel strength G′ (solid lines) between 1,000-2,000 Pa, but the gel broke down at about 10% strain and 15% strain, respectively. See Example 4.

FIGS. 5 and 6 show SEM (300× magnification) photograph for cellulose-containing residue particles A and B, respectively, as described in Example 8.

FIG. 7 shows an SEM (300× magnification) photograph of an acetone-dried cellulose-containing residue of the invention having very fine and porous features that were discernable within the agglomerated structure. See Example 9.

FIGS. 8 and 9 show SEMs for the homogenized, spray-dried cellulose-containing residue of the invention having a very distinct fine particulate structure. FIG. 8 is at 305× magnification and FIG. 9 is at 2000× magnification. See Example 9.

FIG. 10 shows drip weight of two frozen dairy desserts over two hours. Test 1 in FIG. 10 is Sample #1 from Example 10, and Test 2 in FIG. 10 is Sample #2 from Example 10.

### DETAILED DISCUSSION OF THE INVENTION

The process of the present invention comprises purifying a cellulose-containing seaweed residue after the carrageenan has been extracted from the seaweed material.

In a typical carrageenan manufacturing process, crude carrageenan-containing seaweed is first washed with cold water to remove sand and other particulates that may be present after the seaweed has been harvested. Carrageenan typically does not swell during the cold wash, primarily because carrageenan in seaweed is associated with the structural components of the seaweed, generally cellulose. This washing step may be done in the present invention prior to the extraction step in step (i).

Carrageenan may be extracted from the carrageenan-containing seaweed material in the present invention by using a hot aqueous treatment wherein the hot aqueous treatment is an aqueous solution comprising all water or water with other components that may be typically used such as alkali or alkaline earth metal components. After optionally washing in cold water, the seaweed may be placed in an aqueous solution and heated for a time and at a temperature sufficient to solubilize greater than 50% of all carrageenan in the seaweed material. Such conditions may include heating to greater than 60° C., more specifically, from 60 to 140° C., for greater than 30 minutes, more specifically, about 30 minutes to 8 hours, about 30 minutes to 6 hours or about 30 minutes to 4 hours. This results in carrageenan dissolving into the water and being extracted (e.g., by filtering) from the seaweed.

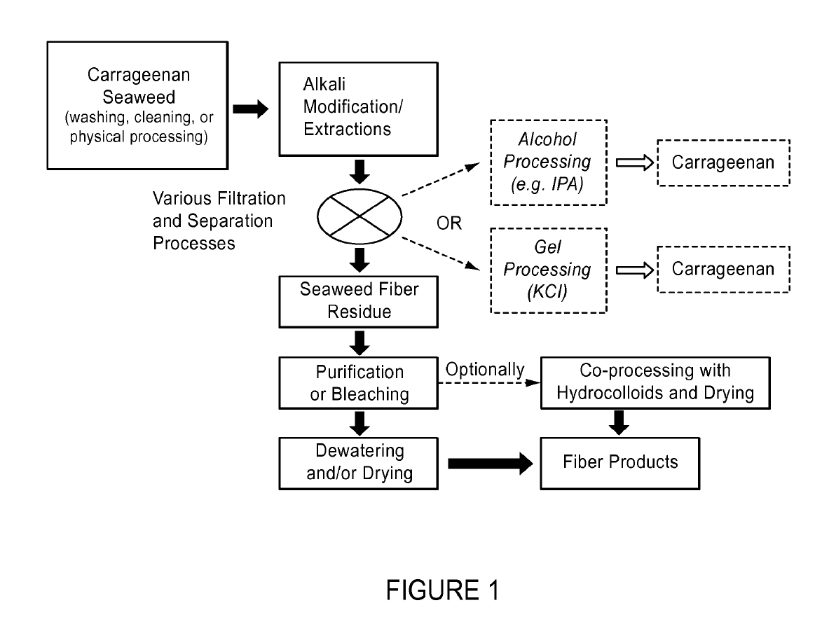

As discussed above, in some instances, for example, with Eucheuma spinosum and Eucheuma cottonii, the hot aqueous treatment also contains an alkali or alkaline earth metal hydroxide such as, for example, NaOH, Ca(OH)₂, or KOH in sufficient quantities (e.g., from 0.1% to 20% by weight of the seaweed) to modify the carrageenan (transforming the D-galactosyl 6-sulfate units into 3,6 anhydro-D-galactosyl units) (as defined herein, the “conventional extract process”). See FIG. 1.

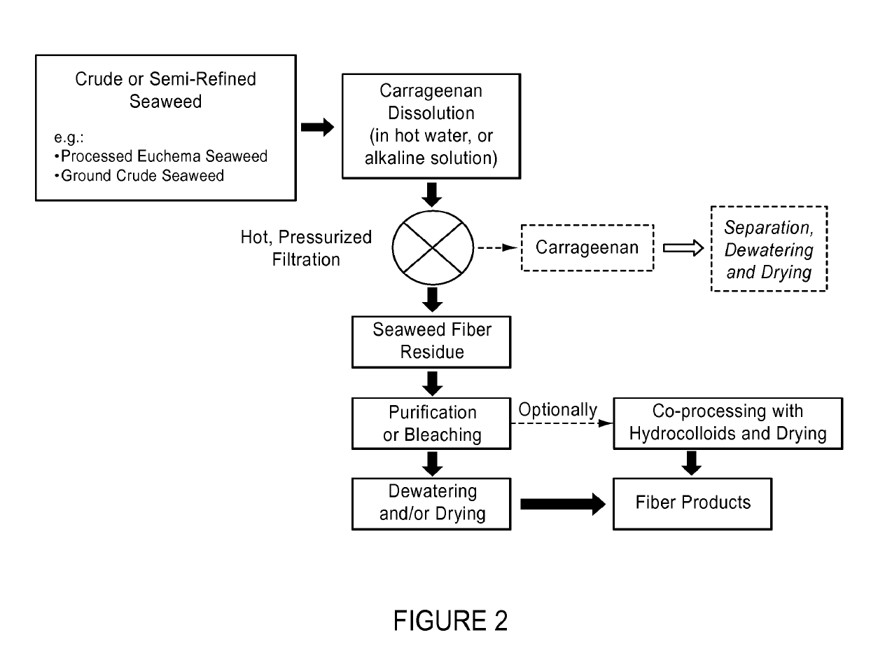

The hot water or hot alkali extraction may also be applied to (or incorporated into) the semi-refined carrageenan (SRC) process, wherein the seaweed had been previously processed, for example, with KCl or NaCl and/or an alcohol such as isopropanol. See FIG. 2. Examples of SRC processes include those set forth in U.S. Pat. No. 6,479,649; incorporated herein by reference.

The carrageenan-containing seaweed material of the present invention comprises any carrageenan-containing seaweed material such as seaweed from the families of Gigartinaceae, Hypneaceae, Solieriaceae, Phyllophoraceae and Furcellariaceae and mixtures thereof. Useful genera include Chondrus, Iridaea, Gigartina, Rhodoglossum, Hypnea, Eucheuma, Agarchiella, Gymnogongrus, Phyllophora, Ahnfeltia and Furcellaria and mixtures thereof. Useful species include Eucheuma spinosum, Eucheuma cottonii, Chondrus Crispus, Gigartina skottsbergii and mixtures thereof. When used in the present invention, the carrageenan-containing seaweed may be crude or washed, wet or dried, in whole form or chopped, milled or ground.

The extraction step in the present invention typically removes ≧50% by weight of all the carrageenan in the carrageenan-containing seaweed material; more specifically, ≧60%, ≧70%, ≧80%, ≧90%, ≧95% and ≧99%. This results in the cellulose-containing residue containing ≦50% by weight of carrageenan based on the total starting weight of the carrageenan in the carrageenan-containing seaweed material; more specifically, ≦50%, ≦40%, ≦30%, ≦20%, ≦10, and ≦5%. Depending on the extraction and purification steps, the carrageenan remaining in the residue before or after the purification step could be 0% to less than 50% by weight of the cellulose-containing residue, more particularly, 0% to 30%, 0% to 25% by weight of the cellulose-containing seaweed residue.

After the extraction step, the cellulose-containing seaweed residue of the present invention will contain the cellulose of the invention, as well as other possible components such as hemicellulose (e.g., xylans and mannans) and galactans such as whatever minor amounts of carrageenan, if any, that might remain after the extraction step. More specifically, after the purification step, the cellulose contained in the cellulose-containing seaweed residue may be present in an amount of greater than 25% by weight of the residue, more specifically, in an amount of from 25-100% by weight of the cellulose-containing residue, more specifically, greater than 50%, greater than 60%, greater than 70%, greater than 80%, greater than 90%, greater than 95%, greater than 99% by weight of the cellulose-containing seaweed residue. The amount of the cellulose in the cellulose-containing seaweed residue will typically be greater when the purification step includes the hydrolysis step.

In addition, after the purification step, the amount of any hemicelluloses (such as xylans and mannans) may be from 0-30% by weight of the cellulose-containing seaweed residue, more specifically, 0-10% of the cellulose-containing seaweed residue, and the amount of any galactans, including any carrageenan remaining in the cellulose-containing seaweed residue after the extraction step, may be from 0-30% by weight of the cellulose-containing seaweed residue, more specifically, from 0-10% by weight of the cellulose-containing seaweed residue.

In general, the amount of any cellulose that may be recovered will vary depending on the species. For example, Eucheuma species such as Eucheuma cottonii and Eucheuma spinosum may generate a cellulose-containing seaweed residue containing greater amounts of cellulose than those residues from other species such as such as Chondrus crispus, Gigartina species, and Furcellaria species. With respect to SRC processes, Eucheuma cottonii has been reported to contain 9-15% Acid Insoluble Matter (AIM) and Eucheuma spinosum has been reported to contain 6-10% AIM. AIM in SRC, which is measured as the residue after 1% H₂SO4 acid hydrolysis of the phycocolloids, is about ˜90% cellulose.

The hydrolysis and bleaching steps may be any that are well known in the field. Examples of hydrolysis include acid hydrolysis which effectively removes, for example, other polysaccharides. The amount of acid used in the hydrolysis step can range from 0.1% to 20% based on the weight of the cellulose-containing residue, preferably, 0.2% to 10%, more preferably, 0.2% to 5%. The bleaching step may utilize bleaching agents such as hydrogen peroxide, peroxyacids, persulfates, organic peroxides, hypochlorite, or ozone. Hydrogen peroxide is a preferred bleaching agent. The amount of bleaching agent may range from 0.2% to 40% based on the weight of the cellulose-containing seaweed residue, preferably from 0.5% to 30%, and 0.5% to 20%. The temperature of the bleaching step may range from 30° C. to 120° C., preferably from 40° C. to 100° C. Preferably, the bleaching is done under alkaline conditions such as a pH>7.

After purification, the cellulose-containing seaweed residue may optionally be dewatered and/or dried. Drying of the cellulose may be achieved by: solvent drying, spray drying, air-drying, fluid bed drying, flash drying, drum drying, belt drying, tray drying, or bulk drying. Solvent drying and spray drying are particularly preferred. Dewatering the cellulose-containing seaweed residue may be achieved by conventional methods such as pressure filtration, batch/continuous centrifugation, press filtering, belt pressing, screening, drum filtering, or flotation. The dewatering step may further use a cationic flocculent or filtering aid as desired.

The purified cellulose-containing seaweed residue obtained from the present invention may be coprocessed with a hydrocolloid. Such hydrocolloids, while not limiting, include guar gum, konjac, glucomannan, locust bean gum, xanthan gum, sodium alginate, PGA, PES, carrageenans (e.g., kappa, iota, kappa-2, and lambda carrageenan), furcellaran, agar, sodium carboxymethylcellulose, cellulose ether (such as hydroxypropyl cellulose, hydroxyethyl cellulose, methyl cellulose and hydroxypropylmethyl cellulose), starches or modified starches, pectins, gellan gums, wellan gum, pullulan, beta-glucans, tamarind seed gum, Arabic gum, tragacanth gum, tara gum, cassia gum, and mixtures thereof. Coprocessing, as used herein, means that the cellulose-containing seaweed residue and hydrocolloid is prepared in a manner which produces a substantially homogeneous product, as distinguished from a two component physical mixture (e.g., a dry blend). The coprocessing may be carried out by any effective means which provides a substantially homogenous product and does not result in significant isolation and separation of one of the cellulose-containing seaweed residue or hydrocolloid. Examples of suitable processes include mixing the cellulose-containing seaweed residue and hydrocolloid in water to dissolve the hydrocolloid (the cellulose-containing seaweed residue is water insoluble) followed by coagulation in an aqueous organic solvent such as isopropanol followed by drying. Such drying can be drum drying, spray drying, air drying, fluid bed drying and freezing followed by pressing or drying. Coprocessing includes coprecipitation, coagulation and water phase mixing. The cellulose-containing seaweed residue may also be mixed, e.g., under high shear (and/or elevated temperature) with the dissolved hydrocolloid and dried (such as by spray drying).

The present invention is also directed to the cellulose-containing seaweed residue obtained in the processes of the invention. The inventors have discovered that the cellulose-containing seaweed residue of the invention has unique morphology and functionality. That is, the cellulose-containing seaweed residue of the invention may be a particulate wherein greater than 90% of all the particles in the particulate have a particle size between 1 and 1,000 μm. At least 50%, at least 60%, at least 70%, at least 80% of the particles may have a globular morphology (meaning that at least 25% of the particle is rounded) when viewed under a microscope at 300×. The cellulose-containing seaweed residue has also been found to have a gel strength of over 1,000 Pa at 2% solids in water prior to drying when bleached. In addition, the cellulose-containing seaweed residue of the invention has a water binding capacity ≧200% after spray drying. The cellulose-containing seaweed residue of the invention also has a stable water suspension after drying, e.g., through alcohol evaporation, when bleached. Such morphology, particle size and other properties are heretofore unknown. The amount of cellulose-containing seaweed residue and hydrocolloid in the coprocessed product may be determined based on the desired functionality, but generally may be from 2 to 80% hydrocolloid based on the total weight of cellulose-containing seaweed residue and hydrocolloid.

Microcrystalline cellulose is a white, odorless, tasteless, relatively free flowing, crystalline powder that is virtually free from organic and inorganic contaminants. It is a purified, partially depolymerized cellulose. It is a highly crystalline particulate cellulose consisting primarily of crystalline aggregates which are obtained by removing amorphous (fibrous cellulose) regions of a cellulosic material. Microcrystalline cellulose is used in a variety of applications including foods, pharmaceuticals and cosmetics, and may specifically be used as a pharmaceutical excipient, particularly as a binder, disintegrant, flow aid, and/or filler for preparation of compressed pharmaceutical tablets.

The cellulose-containing seaweed residue of the invention may be used to make a novel type of microcrystalline cellulose using conventional acid hydrolysis processes. Making microcrystalline cellulose may be accomplished as part of the purification step. For example, the microcrystalline cellulose may be produced by the cellulose-containing seaweed residue of the invention with a mineral acid, preferably hydrochloric acid or sulfuric acid. The acid selectively attacks the less ordered regions of the cellulose chain thereby exposing and freeing the crystalline sites which form crystallite aggregates which constitute the microcrystalline cellulose. These may then be separated from the reaction mixture, and washed to remove degraded by-products. When the wetcake is dried and freed of water the resulting product, a novel type of microcrystalline cellulose is obtained. It is a white, odorless, tasteless, relatively free-flowing powder, insoluble in water, organic solvents, dilute alkalis and acids. See U.S. Pat. No. 2,978,446 for a general description of the manufacturing methods of microcrystalline cellulose.

The microcrystalline cellulose made from the cellulose-containing seaweed residue of the invention may be dry blended or coprocessed with a hydrocolloid such as sodium carboxymethylcellulose and may be fully dispersible, partially dispersible or not dispersible in water depending on the particle size and desired functionality.

The present invention is also directed to products that contain the cellulose-containing seaweed residue (e.g., the microcrystalline cellulose) obtained in the present invention. Examples include food products, pharmaceutical products (including tablets, capsules, etc.,), agrochemical products, consumer product, healthcare products, biomedical products, personal care products, cosmetic products, tissue or towel products, textile products, paper products, diaper fluff products, hygienic products, detergent products, or industrial products. More specific products include ice cream, frozen dairy desserts, edible films, sausage casings, food wrappings, beverages including soy drinks and dairy beverages such as chocolate milk, juice pulps, controlled release products containing drugs or chemicals, cosmetic facial masks and wound dressings.

The cellulose-containing seaweed residue (e.g., the microcrystalline cellulose) obtained in the present invention might be used in a product as a juice pulp fiber, a dietary fiber, moisture binding agent, moisture management agent, food texturizer, fat replacement, thickener, suspension aid, bulking agent, oil/flavor carrier, encapsulating media, fish oil or krill oil carrier, food extrusion aid, cheese processing binder, tablet binder, anti-caking powder, filler or binder in meat or meat injections, or fiber in bakery food.

As noted above, the cellulose-containing seaweed residue forms stable aqueous suspensions when, e.g., it is bleached. These stable suspensions are typically prepared by adding the cellulose-containing seaweed residue to an aqueous solution (e.g., 0.5-2.5% residue based on the total weight of the suspension) and heating (e.g., >80° C.) and mixing (e.g., in a high shear mixer such as a blender) for sufficient time to generate a stable suspension; e.g., a suspension where no visual phase separation is observed at room temperature (e.g., 20° C. to 23° C.) for at least one day, three days, five days, ten days, three months, six months or one year.

The invention will now be described with respect to certain examples which are merely representative of the invention and should not be construed as limiting thereof. Unless otherwise indicated herein, all parts, percents, ratios and the like are by weight.

### EXAMPLES

### Example I

Eucheuma spinosum seaweed was subjected to hot alkaline modification as the carrageenan extraction step, wherein the carrageenan was dissolved and separated from the seaweed and the cellulose-containing residue was obtained. The cellulose-containing residue was then collected using filter screens and had a solids level of 7.3% by weight of the residue. This residue was bleached with 15% hydrogen peroxide based on the dry weight of the residue, at 85° C. for 1 hour. The bleached cellulosic residue was then coagulated with 75% by weight isopropanol in water. It was then thickened on a screen, dried with the solvent evaporation, and then gently ground into powders. BET measurement indicated that the dried cellulose-containing seaweed residue had a surface area of 1.64 m²/g before grinding, and 2.4 m²/g after gentle grinding (that broke apart the fiber flocs), which was unexpectedly comparable (or slightly higher than) to the commercial wood-based microcrystalline cellulose binders (Avicel® PH 101, at about 1 m²/g). The dried and ground cellulose-containing seaweed residue had an average particle size of 135 microns, as measured by a Horiba LA-910 laser scattering particle size distribution analyzer. This was also significant as the particle sizes were comparable to some commercially available microcrystalline cellulose (wood based) particle sizes. This result is unexpected because the cellulose-containing seaweed residue of the invention was not subjected to the acid hydrolysis step utilized in microcrystalline cellulose processes and yet it had comparable surface areas. These findings indicate that the cellulose-containing seaweed residue obtained in the invention (i.e., from heretofore waste material) possesses commercially significant and important functionality.

### Example 2

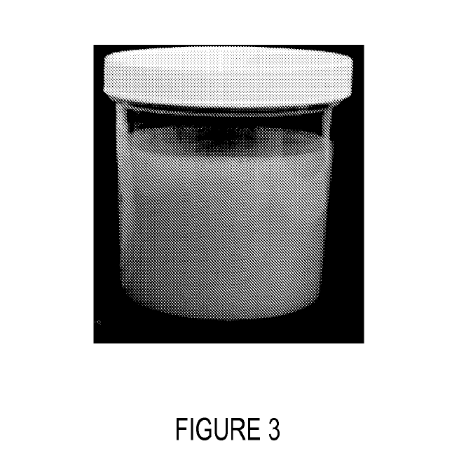

The cellulose-containing seaweed residue obtained in Example 1 was dispersed at room temperature (e.g., 20-23° C.) in deionized water in a Waring blender at 2.6% solids and formed a complete suspension. All the cellulose-containing seaweed residue was fully dispersible in water at room temperature (20-23° C.) without any precipitation. The suspension had an initial Brookfield viscosity of 132 cps and a set-up viscosity after 24 hours of 900 cps (when measured at 20 rpm at room temperature (about 20-23° C.). Rheological tests showed that the suspension was shear-thinning and had an apparent gel strength G′ of 3 Pa. See FIG. 3. The rheological properties and gel strength were surprisingly similar to commercially important colloidal microcrystalline cellulose products that are coprocessed with carboxymethyl cellulose.

### Example 3

Eucheuma spinosum seaweed was subjected to hot alkaline modification as the carrageenan extraction step, wherein the carrageenan was dissolved and separated from the seaweed and the cellulose-containing seaweed residue was obtained. The cellulose-containing seaweed residue was collected after carrageenan extraction. In one batch, this residue was coagulated with 75% isopropanol in water, passed through screens, dewatered, and solvent dried overnight. No hydrolysis or bleaching of the residue in the first batch was performed. The recovered cellulose was dark colored, and had a compact structure. In a second batch, the cellulose-containing seaweed residue was bleached with 15% hydrogen peroxide (based on the weight of the residue) at 92-95° C. for 2 hours. It was then coagulated with 75% strength isopropanol, screened, dewatered, and solvent dried. The recovered cellulose-containing seaweed residue had a fluffy powder structure and was much brighter in color than the unbleached residue in the first batch. An alkaline pressure filtration method was used to estimate the remaining carrageenan content in the bleached cellulosic residue of the second batch and showed <0.5% of carrageenan by weight of the residue.

A hot activation procedure was used to evaluate the rheological properties of these two residues. Both samples were heated to 89° C. for 5 minutes at 1.0% solids in a Thermomixer and then mixed at high shear for 2 minutes in a Waring blender. For the bleached cellulose residue, an initial Brookfield viscosity at 75° C. was found to be 750 cps and the viscosity became 3,950 cps after 24 hours and cooled to room temperature. The hot activated suspension of the bleached fiber was very stable. For the unbleached fiber, however, the initial viscosity at 75° C. was only 4 cps, and upon cooling to room temperature, the cellulose residue had severe phase separation without forming a stable suspension.

### Example 4

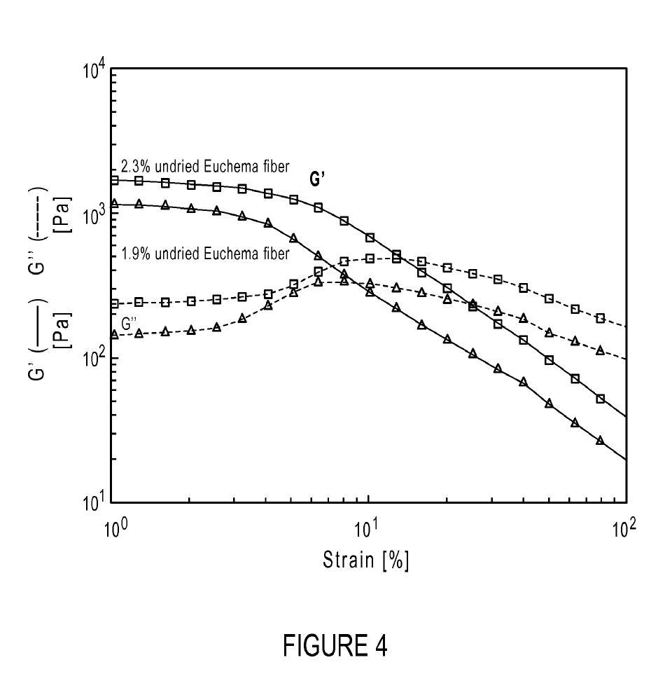

Eucheuma spinosum seaweed was subjected to alkaline modification as the carrageenan extraction step, wherein the carrageenan was dissolved and separated from the seaweed and the cellulose-containing seaweed residue was obtained. The cellulose-containing seaweed residue was collected after carrageenan extraction. This cellulose-containing seaweed residue was bleached with 15% hydrogen peroxide in a glass-lined pilot reactor, at 93° C. for 1.5 hours, and then washed by extensive water and centrifuged to 5.6% solids in water. It was too thick to be measured for viscosity and rheology. Dilution of this bleached cellulose to 2.3% solids in water showed a Brookfield viscosity of 12,000 cps (when measured at 20 rpm at room temperature (about 20-23° C.)). Dilution of this bleached cellulose to 1.9% solids showed a Brookfield viscosity of 5,600 cps (when measured at 20 rpm at room temperature (about 20-23° C.)). The average particle size for these two samples was 112.7 micron and 96.6 microns, respectively, as measured by the Horiba-LA-910 laser scattering particle size distribution analyzer. Rheology test of the 1.9% and 2.3% solids cellulose suspensions using a Texas Instrument rheometer (measured in oscillating mode as a function of strain) showed an unexpectedly very high gel strength G′ between 1,000-2,000 Pa, but, as seen in FIG. 4, the gel broke down (e.g., see the intersecting points between G′ (elastic modulus) and G″ (loss modulus)) at about 10% strain and 15% strain, respectively. The 2.3% solid suspension is represented in FIG. 4 by the square lines and the 1.9% solid suspension is represented in FIG. 4 by the triangle lines. In FIG. 4, G′ is represented by the solid lines and G″ by the broken lines. The bleached cellulose was substantially brighter in color than the unbleached cellulose. Also, it is important to note that these roughly 2% suspensions (i.e., about 98% water) formed gels having a high water holding ability as illustrated by the fact that both suspensions were capable of being turned upside down in a flask and remained at the top of the inverted flasks for several months.

### Example 5

The undried, bleached, washed, centrifuged, Eucheuma cellulose-containing seaweed residue made in Example 4 was diluted in water at ˜2% solids, homogenized at 3,000 psi, and spray dried into powder form. Unlike the solvent dried residue obtained in Example 4, the spray dried cellulose powder in this Example could not be stably suspended in water at 2.6% solids at room temperature. All the cellulosic particles precipitated out. Water binding capacity tests showed the spray dried cellulose had a value of 210%, which is significantly and unexpectedly higher than the non colloidal microcrystalline cellulose made from wood pulp (such as Avicel® PH 101) values of around 170 g water/g cellulose. This difference is important and indicates that the cellulose of the invention may be used to substitute for commercial non-colloidal microcrystalline cellulose powders, for instance, in food or pharmaceutical microcrystalline cellulose applications when suspensions are not desired. The average particle size of the cellulose in this batch was 12.2 microns, as measured by a Horiba-LA-910 laser scattering particle size analyzer.

### Example 6

The purpose of this example was to obtain a non-dispersible, cellulose-containing seaweed residue of the invention. Eucheuma spinosum seaweed was subjected to hot alkaline modification as the carrageenan extraction step, wherein the carrageenan was dissolved and separated from the seaweed and the cellulose-containing residue was obtained. It was then bleached with 15% hydrogen peroxide at 75° C. for 1 hour. The bleached fiber was neutralized, and centrifuged. It was then homogenized, spray-dried and ground. It had an average particle size of 22.0 micron as tested by a Horiba la-910 laser scattering particle size distribution analyzer. When re-dispersing the powder at room temperature into water at 2.6% solids, this batch of spray-dried fiber was originally suspended, but it soon started to precipitate. By overnight, the cellulose residue had completely settled.

### Example 7

Coprocessed Products—The bleached, washed, and centrifuged Eucheuma spinosum cellulose-containing seaweed residue obtained in Example 4 was placed in 5.6% solids in water and then further diluted and blended with other hydrocolloids as noted below. Six coprocessed blends were prepared: 1) 90/10 cellulose residue/guar gum; 2) 90/10 cellulose residue/SRC kappa carrageenan; 3) 90/10 cellulose residue/xanthan gum; 4) 90/10 cellulose residue/CMC Aqualon 12M31P; 5) 90/20 cellulose residue/CMC Aqualon 7Lf; and 6) 70/30 cellulose residue/sodium alginate (Protanal LF200FTS). The homogeneous mixtures were then spray dried at 2-3% solids onto a metal wall surface and acceptable stand alone films were prepared.

### Example 8

The purpose of this example was to use the cellulose residue obtained in the present invention in a conventional microcrystalline cellulose process. That is, the cellulose residue powder sample made in Example 1 was acid hydrolyzed at 10% HCl, at 100° C., for 0.5 hour to produce microcrystalline cellulose. Particles of two general types were obtained. That is, after the reaction, it was found under light microscope that the filtered wet cellulose cake produced particles approximately 1 μm to 5 μm wide on average (cellulose residue particles A). This is quite different from acid hydrolysis to produce microcrystalline cellulose from wood pulp, under the same conditions, which was found to have average dimensions of approximately 50 μm to 100 μm in length and 15-50-μm in width depending on the wood source. The microcrystalline cellulose particle A made from the cellulose in Example 1 was dried in the oven at 75° C. and ground into a powder. BET surface area test by a Micrometrics Tri-Star 3000 indicated a value of 1.4 m²/g, which was in the general range of commercial microcrystalline cellulose made from wood pulp.

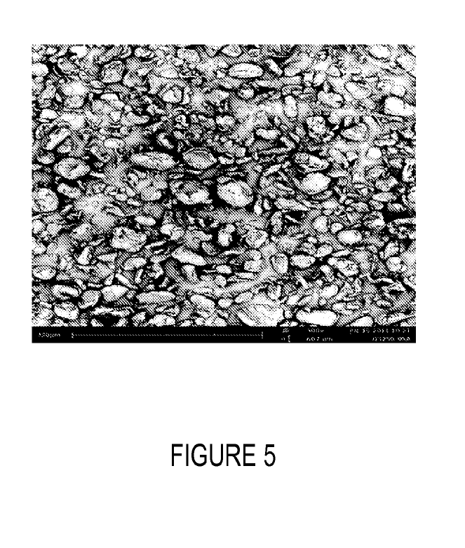

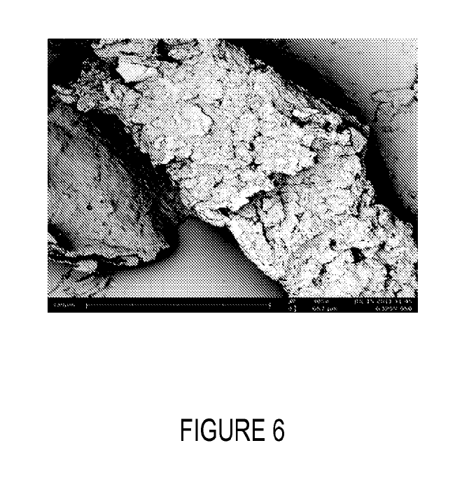

In addition to the above cellulose residue particles A, the acid hydrolysis also produced extremely fine particles (cellulose particles B) which could only be recovered on the filter paper and formed a transparent film on the paper. A scanning electron microscopy (SEM) observation of these two different cellulose particles was taken to show the fine texture/morphology of the aggregated state of each sets of particles at 300×. Cellulose particles A are shown in FIG. 5 and cellulose particles B are shown in FIG. 6. The shapes of the cellulose particles A were round and globular. On the other hand, cellulose particles B of the invention were agglomerated into very porous structures after drying. Both particles A and B of the invention can be used, for example, in food and pharmaceutical applications.

### Example 9

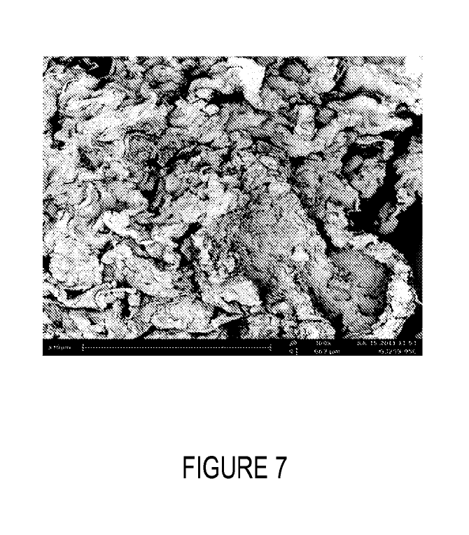

Eucheuma spinosum seaweed was subjected to hot alkaline modification as the carrageenan extraction step, wherein the carrageenan was dissolved and separated from the seaweed and the cellulose-containing seaweed residue was obtained. It was then bleached with 15% hydrogen peroxide at 75° C. for 1 hour. The bleached seaweed residue was then neutralized, washed with water, and then washed with acetone, filtered, and air-dried. Under SEM (300× magnification), the acetone-dried cellulose had very fine and porous features that were discernable within the agglomerated structure. See FIG. 7. This agglomerated structure is significantly different than the structure of the cellulose obtained from wood.

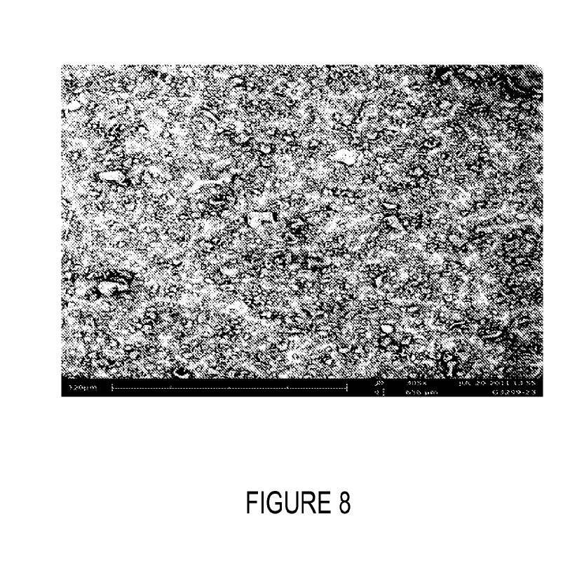

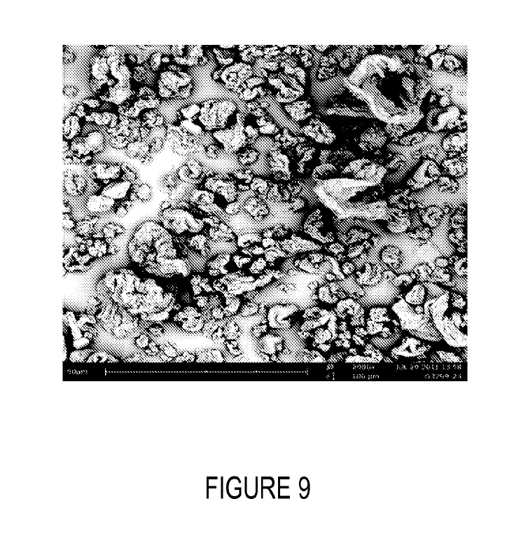

In distinction to microcrystalline cellulose from wood sources, the homogenized, spray-dried cellulose residue of the invention had a very distinct fine particulate structure. See FIGS. 8 (305× magnification) and 9 (2,000× magnification). As a result, it can be seen that the morphology of the cellulose residue of the present invention is significantly different than the morphology of the cellulose obtained from other sources of cellulose such as wood and agricultural material.

### Example 10

Cellulose-containing seaweed residue of the invention was evaluated in a frozen dairy dessert formulation and compared directly against a commercially available microcrystalline cellulose based stabilizer (that is, Gelstar® XP 3623 which is a microcrystalline cellulose from a wood source). Microcrystalline cellulose from wood sources is well known for its ability to impart creamy eating quality to ice cream products as well as reduce the occurrence of large ice crystal formation resulting from temperature abuse. In addition, microcrystalline cellulose from wood sources is also known to reduce the rate at which ice cream melts. All aspects of the process and product attributes were tested in this example including mix viscosity, freezing properties, eating quality, and melt down observations.

The base formulation used in the evaluation was chosen to be a lower quality type product falling outside the standard of identity of ice cream. This is because such lower quality formulations can often be used to better assist in demonstrating the functional differences in various stabilizers. In this example, the butterfat content was 3.5% and the total milks solids nonfat (MSNF) was 12% (66% traditional MSNF, and 34% whey powder). Any formulation where the whey powder exceeds 25% of the total MSNF is considered a frozen dairy dessert. The complete formulation can be found in Table #1.

Both mix formulas were processed using typical HTST processing conditions. Prior to pasteurization, milk and cream were added to a 10-gallon breddo liquefier and brought under mild agitation. The stabilizer powder was added to the liquefier followed by milk solids, corn syrup solids, maltodextrin and cane sugar. The mix was allowed to hydrate for ˜10 minutes prior to pasteurization on a HTST system set to homogenize the mix at 2,500 psi followed by holding a temperature of 183° F. for 25 seconds. Following pasteurization, the mix was immediately cooled to 42° F. and allowed to age overnight.

All products were frozen in a continuous freezer (WCB Model 100) @˜21.5° F. with an overrun of 100%. Once conditions for each mix were established, several pint containers of each variable were collected and placed in a −30° F. blast freezer. Prior to characterizing the samples, 1 pint of each variable was placed in a tempering cabinet @ 0° F. for several hours. Table #1 shows the recipe of the mixes processed in this example. All % in Table #1 are % by weight of the total formulation.

TABLE #1

| | Sample # | |
|---|---|---|
| | 1 | 2 |
| Components | % | % |
| Butterfat | 3.5 | 3.5 |
| MSNF | 8 | 8 |
| Whey Powder | 4 | 4 |
| Sucrose | 11.5 | 11.5 |
| Corn syrup Solids (36DE) | 5 | 5 |
| Maltodextrin M-180**** | 3.25 | 3.25 |
| Microcrystalline Cellulose* | 0.4 | |
| Bleached Seaweed Residue | | 0.4 |
| CMC ** | .175 | .175 |
| Mono & Di glycerides***** | .164 | .164 |
| Carrageenan *** | .01 | .01 |
| Total Solids | 36.0 | 36.0 |

*Gelstar ® XP 3623-MCC/CMC
**Carboxymethylcellulose (Aqualon 7HF)
***Carrageenan FMC Seakem IC 518
****Maltodextrin M-180-(18 DE) GPC
*****Alphadim 70K (Caravan Ingredients)

Each sample was subjected to sensory analysis. Sensory analysis was completed by manipulating a spoonful size of the frozen dairy dessert in the mouth and subjectively determining the structure or body of the mass. Body can be classified as weak, gummy, crumbly, short, fluffy, or soggy. Texture is the other main parameter to consider while completing a sensory analysis. Descriptors of texture include coarseness, iciness, sandy, or greasy. Body and texture descriptors are ranked as either being heavy, moderate, or light. Prior to the sensory evaluations, the frozen dairy desserts from Table #1 were tempered to 5° F.

When evaluated prior to being subjected to temperature abuse (heat shock), various analysts agreed that Test Sample #2 outperformed Test Sample #1 in terms of providing a creamier texture and heavy body. Heat shock abuse was applied to the samples by placing the pint containers in a temperature controlled cycling cabinet programmed to maintain 15° F. for 12 hours followed by cooling to 0° F. for 12 hours. This cycling pattern was repeated for 14 days. Sensory scoring of these samples showed that Test Sample #2 maintained superior body while the size of ice crystals were subjectively determined to be equivalent between the two samples.

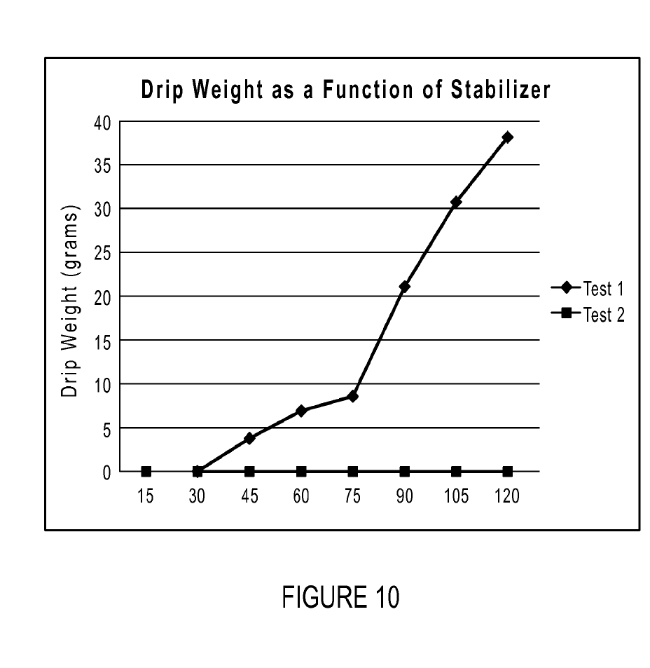

Melt down observations were recorded to determine the effect each stabilizer variation had on the rate at which the product melts. In this test, an 8 oz cup of frozen dairy dessert was placed on a 10-mesh wire screen. As the frozen dairy dessert melts, the weight of the mass that passes through the screen was recorded and plotted in chart form in FIG. 10. The observations were recorded at 70° F. The melt data in FIG. 10 clearly demonstrates the structure of Sample 2 after two hours when compared to Sample 1. Within a period of two hours, the frozen dairy dessert of Sample 2 did not produce any drips and the shape of the thawed frozen dairy dessert for Sample 2 resembled the shape of the frozen mass prior to subjecting it to room temperature.

While the invention has been described in detail and with reference to specific embodiments thereof, it will be apparent to one skilled in the art that various changes and modifications can be made therein without departing from the spirit and scope thereof.

1. A process comprising:
(i) extracting ≧50% by weight of all carrageenan from a carrageenan-containing seaweed material to obtain a cellulose-containing seaweed residue; and
(ii) purifying the cellulose-containing seaweed residue by at least one of hydrolysis or bleaching.

2. The process of claim 1, wherein said cellulose-containing seaweed residue obtained in step (ii) is incorporated into a food or pharmaceutical product.

3. The process of claim 1, wherein said purifying comprises acid hydrolysis to form microcrystalline cellulose.

4. The process of claim 1, wherein said purifying comprises bleaching.

5. The process of claim 1, wherein the carrageenan-containing seaweed material comprises seaweed from the families of Gigartinaceae and Solieriaceae and mixtures thereof.

6. The process of claim 5, wherein said carrageenan-containing material is seaweed from Eucheuma spinosum, Eucheuma cottonii, Chondrus Crispus, Gigartina radula, Gigartina skottsbergii and mixtures thereof.

7. The process according to claim 1, wherein said cellulose-containing seaweed residue obtained in step (ii) is subjected to dewatering or drying.

8. The process of claim 1, wherein the carrageenan-containing seaweed material is subjected to alkali modification prior to step (i).

9. The process of claim 1, wherein said step (i) extracts ≧80% by weight of all carrageenan in said carrageenan-containing seaweed material.

10. The process of claim 1, wherein said step (i) extracts ≧95% by weight of all carrageenan in said carrageenan-containing seaweed material.

11. The process of claim 1, wherein said step (i) extracts ≧99% by weight of all carrageenan in said carrageenan-containing seaweed material.

12. The process of claim 1, wherein said cellulose-containing seaweed residue obtained in step (ii) is coprocessed with a hydrocolloid.

13. A cellulose-containing seaweed residue obtained from claim 1.

14. The cellulose-containing seaweed residue of claim 13, wherein the cellulose-containing seaweed residue is a particulate and at least 50% of the particles have a globular morphology when viewed under a microscope at 300×.

15. The cellulose-containing seaweed residue obtained in claim 4, wherein the cellulose-containing seaweed residue has a gel strength of over 1,000 Pa at 2% solids in water prior to drying.

16. The cellulose-containing seaweed residue of claim 13, wherein said cellulose-containing seaweed residue has a water binding capacity of ≧200% after spray drying.

17. The cellulose-containing seaweed residue obtained in claim 4, wherein said cellulose-containing seaweed residue forms a stable water suspension.

18. The cellulose-containing seaweed residue of claim 13, wherein the cellulose-containing seaweed residue is coprocessed with a hydrocolloid and the hydrocolloid is present in an amount of 2%-80% by weight of the cellulose-containing seaweed residue and hydrocolloid.

19. The cellulose-containing seaweed residue of claim 13, wherein said cellulose-containing seaweed residue is a particulate and wherein greater than 90% of all the particles have a particle size between 1 and 1,000 μm.

20. The cellulose-containing seaweed residue of claim 13, wherein the cellulose-containing seaweed residue is acid hydrolyzed to form microcrystalline cellulose.

21. A product comprising the cellulose-containing seaweed residue of claim 13, wherein said product is a food product, pharmaceutical product, agrochemical product or cosmetic product.

22. The product of claim 21, wherein said food product is ice cream, frozen dairy dessert, edible film, sausage casing, food wrapping, beverage, chocolate milk, soy drink, juice pulp, and said pharmaceutical product is a tablet or capsule.

23. The product of claim 22, wherein said product is an aqueous suspension and said cellulose-containing seaweed residue has been bleached.
